(19) United States
(12) Patent Application Publication
(10) Pub. No.: US 2017/0120037 A1
(43) Pub. Date: May 4, 2017

(54) Iso compliant male luer tapered valves

(71) Applicant: Gale Harrison Thorne, Bountiful, UT (US)

(72) Inventor: Gale Harrison Thorne, Bountiful, UT (US)

(21) Appl. No.: 15/530,257

(22) Filed: Dec. 15, 2016

Related U.S. Application Data
(63) Continuation-in-part of application No. 14/921,343, filed on Oct. 23, 2015.
Application No. 14/921,343 is a continuation-in-part of application No. 14/121,681, filed on Oct. 7, 2014.
Application No. 14/121,681 is a continuation-in-part of application No. 13/872,828, filed on Apr. 29, 2013, now Pat. No. 9,295,827.

(51) Int. Cl. A61M 39/26; F16K 15/14

(52) U.S. Cl. CPC A61M 39/26; F16K 15/147

(57) ABSTRACT

Two embodiments of a normally closed tapered fitting valve are disclosed. Each tapered fitting valve comprises a single molded incompressible, but supple part and a skeletal support whereby the tapered fitting valve is opened by insertion into a female tapered fitting. Use of the valve specifically targets use with medical luer fittings. The preferred embodiment of an actuator portion of the valve is preferably elliptical in shape. The valve opens by compressing a slit which is disposed along a major elliptical axis as it is advanced through a tapered circular duct. A stand-alone male adapter comprising the tapered fitting valve is disclosed. Also, a syringe barrel comprising a skeletal support structure for a securely affixed valve to thereby provide a syringe barrel with an integrally affixed male adapter is disclosed.

### CONTINUITY

This application is a Continuation-in-Part of U.S. patent application Ser. No. 14/921,343 filed Oct. 23, 2015 and titled DUAL-CHAMBER SYRINGE AND ASSOCIATED CONNECTING SYSTEMS by Gale H. Thorne, Jr., et al., (to be referenced hereafter as Thorne 343) which is a Continuation-in-Part of U.S. patent application Ser. No. 14/121,681 filed Oct. 7, 2014 and titled COMPONENTS AND DEVICES FOR CLOSED MEDICAL SYSTEM OPERATION by Gale H. Thorne (to be referenced hereafter as Thorne 681) which is a Continuation-in-Part of U.S. patent application Ser. No. 13/872,828, filed Apr. 29, 2013 and titled TWISTED SLIT VALVE filed by Gale H. Thorne (to be referenced hereafter as Thorne 828), now on record as U.S. Pat. No. 9,295,827 B2, allowed Mar. 29, 2016, contents of each of which are made part hereof, by this reference.

### FIELD OF INVENTION

This application relates to tapered fitting systems which employ self-closing valves, in general, and to male valves opened by insertion into tapered female fittings, in particular, such valves being opened by compressive forces about the exterior of the valve and, once compressive force is removed, be self closing to stop fluid flow. Inventions disclosed within this application also relate, generally, to applications of such valves in male luer adapters for needleless interconnections, male luer replacement by such valves in otherwise conventional syringes. As such luer valves may be used internationally and be regulated by ISO 594-1 which stipulates that a male luer fitting should not exceed 0.0158 inches in diameter at the insertion end.

### BACKGROUND AND DESCRIPTION OF RELATED ART

While the present invention broadly applies to self-closing valves which are opened by insertion into tapered fittings, it has particular application to self-sealing male and female luer valves used, for example, in the following medical applications.

### Example 1: Male Adapter Valves

Two primary prior art patents well disclose the need and opportunity for a male luer valve which is opened upon insertion into a female luer fitting. The first, U.S. Pat. No. 7,766,304 B2 issued to John C. Phillips (Phillips 304) Aug. 3, 2010 and titled, SELF-SEALING MALE LUER CONNECTOR WITH BIASED VALVE PLUG discloses a male luer connector for connection with a female luer connector. Phillips 304 further discloses a device comprising a tubular male body and a surrounding displaceable cuff. A valve plug is slidably disposed within the housing and formed to, in a first state, seal a communicating hole and, in a second state, be displaced to open the hole for fluid communication. Closure is biased to occur by an elastomeric coupling which communicates with the plug.

The second, U.S. Pat. No. 7,803,140 B2 issued to Thomas F. Fangrow, et al (Fangrow 140) Aug. 16, 2011 and titled, MEDICAL CONNECTOR discloses two primary designs for a male luer connector for connection with a female luer connector. The first design comprises a plugging component which is offset to open a valve for fluid flow. The second design discloses a slit valve which is opened for flow by insertion of a piercing part.

Such male valves provide barriers for infecting bacteria and debris, but perhaps more importantly provide a self-closing barrier and, thus, a closed system against inadvertent leakage, wherein product associated with such leakage might be a hazardous drug. It is important to note that such male valves should only be disposed in an open state while the valve is inserted into a complementary female fitting. At this date, all contemporary commercial male adapters known to the inventor for needleless connectors employ either a linear displacement mechanism which removes a “plug” from a hole when the valve is inserted into a female luer fitting or a forceably opened slit. Such mechanisms are commonly complex in structure and, therefore, result in an elevated component cost. Generally within the scope and meaning of this Application, the term male luer adapter shall be used as a reference for such needleless connector devices.

Further, male luer adapters such as those provided as examples, supra, are actuated to an open state by either a displacement of a plug within a hole or by a slit of a valve being parted by insertion of a piercing part. In the case of plug displacement, such is known to often result in a small droplet of liquid remaining resident at the exterior of the hole and plug site upon closure.

### Example 2: Luer Fitting Replacements on Otherwise Conventional Medical Syringes

The value of adding a male adapter fitting to a conventional medical syringe has been demonstrated by at least one company which currently sells one of the above cited male adapters by securely affixing a male adapter to a syringe and selling the combination as an integrated product. As is well understood in medical syringe art, definite advantages in cost and elimination of dead space would result by replacing a male luer fitting on a syringe with a male luer adapter according to the present invention, which replaces a male luer fitting.

Within the scope of this application, terms which are absolute, such as round and unreactive, are understood to be permissive of manufacturing and physical limitations which, while functionally achieving a desired function, do not absolutely comply with definition of each specific term.

### DEFINITIONS FOR TERMS USED

assembly n: a device which is made from at least two interconnected parts
barrel n: a cylindrical elongated portion of a conventional syringe which is substantially of constant diameter along a long axis of the syringe, open on one end to receive a plunger tip and plunger rod assembly used for displacing fluid within the barrel and partially closed at an opposite end except for an orifice or portal through which fluid is ejected or aspirated
conventional adj: sanctioned by general custom; i.e. commonplace, ordinary
disparate n: when used to describe a first volume of contents relative to another volume of contents, the first volume of contents being kept distinctly separate from the other volume of contents
distal adj: a term which depicts placement away from a reference point (e.g. away from a user of a syringe)
downstream adj: a direction which is consistent with flow out of a syringe or away from a user
fluid n: a substance (e.g. a liquid or gas) which tends to take the shape of a container
front adj/n: when referenced to a syringe, distally disposed or a distally disposed site (e.g. a front of a syringe comprises the commonly provided luer fitting and associated orifice)
gas n: a fluid which is neither solid nor liquid
liquid n: a fluid which is neither solid nor gaseous, free flowing like water
medial adj: occurring away from an outer edge; disposed near the center of (e.g. disposed away from an edge or periphery and in the vicinity of a center of gravity or axis of symmetry)
proximal adj: opposite of distal (e.g. a term which depicts placement nearer to a reference point)
state n: a mode or condition of matter, e.g. gaseous, liquid or solid or of a device, such as an open state of a valve
substantially adv: to a most reasonably achievable amount
syringe n: a medical device used for injecting or withdrawing fluids, a syringe usually comprising a plunger and plunger rod disposed to be displaced within a conventional cylindrical syringe barrel and, for a dual-chamber syringe, includes a plunger valve to provide a dual-chamber syringe

### Table 1

### BRIEF SUMMARY AND OBJECTS OF THE INVENTION

In brief summary, this novel invention, while having broader applications, alleviates known problems related to providing a normally closed male tapered fitting valve which is opened when a portion of the valve is inserted into a tapered female luer fitting. Basic to the instant invention is a valve body having asymmetrical side dimensions formed about a planar slit. The valve body is preferably shaped and sized to conform with the width of the slit providing a predetermined, adequate wall thickness from slit to the outer surface of the valve body for device stability and reliability. The valve body is preferably made from a compliant, incompressible material having memory which, when not inserted, reforms to and maintains an unconstrained body in its originally formed (e.g. molded) state. Within the valve body is a normally closed slit providing a valve formed with front-to-back controlled closure about both ends of the slit which provides a common fluid pathway when the valve is opened. The front-to-back closure control, being on opposite ends of the slit, form a normally closed single valve pathway when uninserted. Generally, the body, being asymmetric, is sized and shaped to reform by compression to fit snugly into a tapered female fitting to, thereby, compressively distort the valve body and open a valve pathway when inserted therein. Of course, for medical applications, the material must also be unreactive to physiological fluids. Such a material is butyl rubber which is used in contemporary syringe plunger applications.

In Thorne 828, a twisted slit valve is disclosed. Efficacious operation of the twisted slit valve (i.e. closure to fluid flow upon removal from a tapered female fitting) is highly dependent upon valve slit closure due to twisted geometry and molded material interlinking. The instant invention disclosed herein is free from such constraints by providing valve geometry which is designed to be inherently closed, similar to “duck bill” valve design, when not actuated.

For the case of this instant invention, a pair of normally closed valve lips are preferably formed within that portion of a device which is inserted into a tapered female fitting. Duckbill valves are particularly well adapted for such purposes although a conventional duckbill valve will not meet requirements of a bidirectional barrier. Commonly a duckbill valve has a pair of lips which are closed from pressure in an upstream direction, but open due to pressure exerted in an opposite direction. Providing a duckbill valve having opposition to flow in opposite directions assures valve closure unless the geometry of valve lips are physically (compressively) compromised to open a common communicating pathway.

For a valve which is opened by insertion into a tapered female fitting to operate efficaciously, a number of specific constraints must be overcome. One of the major constraints is associated with circumference compliance. At the distance where the device is fully and sealingly inserted into an associated female fitting, the outer surface of the inserted device should have the same (sealing) circumference as the internal surface of the tapered female fitting along its inserted length. Also, for compressive forces to accomplish an effective seal, cross sectional area of the inserted fitting plus area of a predefined open pathway should be nearly equal the internal cross sectional area of the female tapered fitting along the insertion length. For such a combination to work, the fitting, before being inserted, must be non-circular (asymmetric) yet have the geometric dimensional characteristics previously disclosed.

For small valves, such as valves for luer fittings, dimensional constraints are challenging. To slit a valve while assuring tight maintenance of valve part accuracy, a careful technique for valve production and slitting is highly recommended. As an example, inner diameter of a female luer fitting is nominally less the 0.2 inches and lips of a slit valve may need to be less than 0.02 inches thick. To provide parts which can be effectively and efficiently manufactured, a process which molds and slits a valve before displacing critical mold parts away from the mold may be preferred.

Similar to the twisted valve of Thorne 828, valve opening may be accomplished in either of two modes. The first mode is by compressive distortion of the body to deform the slit from a generally planar state to a more compact hollow cylindrical state, thereby creating an open fluid pathway. As the slit is disposed along a common plane within the valve, a hollow tubular cannula can be displaced through the planar pathway to provide a path for fluid flow, thereby changing the valve to an open state. Note, that, in either case restructuring the body from a compressed state or removing the hollow tubular object should result in automatic lip and, therefore, valve closure.

In the case of valve opening by body distortion, the exterior surface circumference of each body crosscut segment about the slit can be formed to have a predetermined dimension, as disclosed hereafter. Likewise, the dimensions of each valve body crosscut segment will have a predetermined length and width, dependent upon slit length upon which a crosscut circumference conforms. As stated supra, the valve body is preferably designed such that the crosscut circumference is equal along its length to the associated interior surface circumference of a hollow tapered tube (e.g. a female luer fitting) in which the valve is displaced for opening.

In general, a valve device body (e.g. of a male luer adapter), according to the instant invention, has two ends. At the first end, comprising the slit, an asymmetric valve, is formed to be used as a fitting element of a tapered releasible connector. The second end comprises a means for forming a communicating, connecting part whereby fluid may be displaced through the valve. If, for example, the slit valve is part of a stand alone male luer adapter, such as those used in common medical applications, the first end would serve as a male luer fitting while the second end may be formed to provide a female luer fitting having a portion which is attachable to a fluid source implement. In such a case, as the male luer fitting portion of the device is inserted into an associated female luer fitting, flexibility of the slit valve causes material to be distorted while conforming to the inner circumference of the female luer fitting, resulting in formation of a through hole along the path of the slit and thereby opening the valve. It should be obvious to those skilled in incompressible materials art that the circumference of each cross section of the valve should be of the same dimension as the circumference of the cross section of the associated interior of an associated female luer fitting when the valve is fully inserted into the fitting to assure a good, sealing fit.

In addition to being used within a stand-alone male luer adapter, using such an asymmetrically formed slit valve as a replacement for a male luer fitting of a syringe provides a basis for closed valve operation in a plurality of medical syringe applications. Such a replacement can provide a syringe which has a closed fluid delivery orifice which remains closed until the male luer adapter is displaced into a female luer fitting, such as a fitting on an IV set or on a medical needle.

For an internally disposed valve to be forced closed when not inserted, it is well known in the duck bill valve art to apply pressure upon the exterior of the lips about the slit. Such may be accomplished in the instant invention by offsetting one or more dead-ended slits, each forming a blind cavity. Each cavity is designed to apply closing force upon lips of the interior valve.

It should be noted that offsetting a slit as a closure abetting cavity is uniquely different from valve geometry disclosed in patent applications from which this U.S. patent application continues. A previous application disclosed a molded cavity disposed above a proximal portion of the slit (main slit) which is opened to provide a communicating pathway. It has been found that compressive distortion of a molded cavity, especially in a valve having an offset slit, distorts cavity boundaries which are then directed toward the main slit to deter that slit from opening. Also, due to molded cavity dimension requirements, it was found desirable to offset the main slit from the medial axis of the valve. However, with the instant invention, offsetting the main slit is not required. Elimination of the offsetting requirement provides much needed space for providing superiorly and inferiorly disposed blind slits about the main slit in one embodiment. In one embodiment (referenced hereafter as the other embodiment), a pair of blind cavities are formed as a natural consequence of interface with an appropriately formed insert support, as disclosed in detail hereafter.

However, providing such blind cavities require additional slitting, which may be difficult and costly. A preferred embodiment requires but the single slit which is deformed to provide an open valve fluid pathway. Sensitive to maintaining a closed valve against upstream pressure internal to the valve, slit closure can be maintained by communicating the upstream pressure along the plane of the slit. In the preferred embodiment, a pathway for such a purpose is provided by molding grooves to form a channel in the valve face which interfaces with the insert support. Within the channel, fluid communicated from an upstream pressurized source is directed radially outward about the plane of the slit. Such directed pressure causes any valve body expansion to stretch along the slit plane and thereby force the slits more tightly closed.

Another novel and important difference between disclosures of U.S. patent applications from which this instant invention continues are formation of a distal end of the valve which is sized and shaped to meet ISO standards and an elliptically shaped skirt which forms the proximal end of the valve. Entry dimensions of a male luer insert are limited by ISO standards to dimensions which cannot be met directly by an asymmetric valve which is compressively distorted to an open state in a female luer fitting.

For this reason, a diminished front surface of the valve is provided which meets ISO requirements. In the other embodiment, a transition section from the round face to the asymmetric (currently elliptical) body of the valve comprises linear translation dimensions which maintain corresponding circumferential dimensions of a female luer fitting into which the valve is inserted to open. A blind hole, corresponding to opened valve flow dimensions is provided in the front face. This blind hole diminishes in size similar to a half pillow, and because the main slit also is formed through the face and transition section, a flow path consistent in diameter with the flow path of the rest of the valve is opened by compression as the valve is displaced into a female fitting. Due to compression of the transition section fully opens the hole.

In the preferred embodiment, the ISO requirement is met by filleting the front face of the valve to reduce major axis dimensions to be equal to or less than ISO limiting values. It should be noted that the preferred embodiment does not require the open blind hole of the other embodiment.

The elliptical skirt is preferably designed to extend proximally with the same exterior linear dimensions and taper as a female luer fitting into which the valve is displaced for opening. The skirt preferably has a constant skin thickness and is sized and shaped to extend linearly from the valve to a retaining ring at its proximal end. The inner surface of the skirt has circumferential dimensions which are the same as a round, tapered cylindrical support which is inserted into the valve skirt for insertion strength and for defining a fluid sealing, circular female surface contact with a female luer fitting. The cylindrical support comprises a through hole which provides fluid communication from an upstream fluid source and may be a part of a male adapter or of a syringe as disclosed hereafter. For this purpose, the insert support comprises a tapered cylindrical shape having the same circumferential dimensions as corresponding inner surface circumferences of the skirt.

At the interface where the internal support and proximal end of the valve meet, the smaller minor axis of the valve ellipse is understandably smaller than the radius of the circular support. At this interface, the skirt is distorted to correspond with the insertion support radius. This distortion can result in an opening of the blind slits disclosed supra as disclosed for the other embodiment.

Such distortion may also provide forces which act upon the valve slit to force the valve slit partially open. Such is alleviated by providing a fillet on the distal surface of the insertion support which reduces the effect of opposing dimensions at the interface, thereby suppressing inadvertent valve opening.

In the preferred embodiment without blind slits, the fillet provides a sealed section orthogonal to the slit (along the minor ellipse axis). A groove in the intersecting plane of the valve part, about the plane of the slit, provides a pathway communicating to natural cavities formed along the distal ends of the major axis of the elliptical valve which, when acted upon by increased pressure tends to expand the valve body and thereby draw lips of the valve together to assure valve lip closure maintenance.

Accordingly, it is a primary object to provide a normally closed asymmetric, tapered fitting valve which is made from a material which is incompressible, elastic and deformable to be compressibly opened when displaced into an elongated, tapered tube having an inside diameter which conformably deforms the valve to open a fluid pathway along a medially disposed slit.

It is a very important object to provide a normally closed asymmetric, tapered fitting valve comprising but a single molded part which can be affixed as a male luer fitting and opened by displacement into a female luer fitting

It is equally as important to provide an asymmetric, tapered fitting valve for a luer fitting which complies with ISO specifications.

It is an important object to provide a normally asymmetric, tapered closed and self-sealing slit valve which has two distinct and independent opening modes (i.e. by displacement into a hollow tapered tube of predetermined internal surface circumference and by displacement of a blunt cannula through the valve).

It is an object to provide an asymmetric, tapered fitting valve which is displaced to an open state by application of a medially directed force causing compressive deformation when displaced into a circular, tapered fitting.

It is another object to provide a self-sealing asymmetric, tapered fitting valve having a body which is molded from an incompressible, flexible and compliant material which returns to a stable closed state when removed from compression by insertion into a tapered fitting.

It is an object to provide a method for making a common slit pathway through a slit valve which has two opposing normally closed ends within a single molded asymmetric, tapered fitting valve body.

It is an object to provide a stand-alone male luer adapter device which employs the tapered fitting valve.

It is another object to provide a normally closed valve which is formed as an integral part of a medical syringe barrel which is opened by insertion into a female luer fitting.

It is a very important object to provide a closed medical syringe which is normally closed by an integral asymmetric valve integrally affixed to a barrel of the syringe and only opened for fluid flow therefrom by compressive insertion of an integral valved male luer fitting adapter into a female luer fitting.

It is a primary object to provide an asymmetric, tapered fitting male valve which can be used with conventional luer fittings and associated medical syringes.

It is a meaningful object to provide a syringe barrel which comprises a skeletal inertion support for an asymmetric, tapered fitting valve which replaces a male luer fitting conventionally disposed on a syringe such that an insertion support of the syringe barrel and associated valve part provide a male luer fitting which only opens when disposed within a female luer fitting.

It is another meaningful object to provide a male luer adapter as a separate component which employs structure of the tapered fitting valve.

It is an object to provide a cap and associated fitting geometry for a capping and thereby protecting a male luer fitting comprising an asymmetric exterior surface.

These and other objects and features of the present invention will be apparent from the detailed description taken with reference to accompanying drawings.

### BRIEF DESCRIPTION OF THE DRAWINGS

FIG. 1 is a perspective of a preferred embodiment of an asymmetric valve part which is compressively opened according to the instant invention, the valve part being preferably molded from incompressible, elastic material.

FIG. 1A is a perspective of another embodiment of an asymmetric valve part which is compressively opened according to the instant invention, the valve part being preferably molded from incompressible, elastic material.

FIG. 2 is a perspective of the valve part seen in FIG. 1 with shading removed for a clearer view of planes disposed to identify crosscuts of the valve part at predetermined sites.

FIG. 2A is a perspective of the valve part seen in FIG. 1A with shading removed for a clearer view of planes disposed to identify crosscuts of the valve part at predetermined sites.

FIG. 3 is a cross section of a preferred embodiment of the valve part, seen in FIG. 1, in a first radial orientation.

FIG. 3A is a cross section of the valve part seen in FIG. 3, but rotated ninety degrees about a longitudinal axis.

FIG. 3b is an elevation of the distal end of the valve part seen in FIG. 3A.

FIG. 4 is a cross section of an insertion support which is an integral part of a tapered fitting valve made according to the present invention.

FIG. 4A is an elation of the proximal face of the insertion support seen in FIG. 4.

FIG. 5 is a cross section of the insertion support seen in FIG. 4 fully inserted, in a first rotational orientation, into the valve part seen in FIG. 3.

FIG. 5A is a cross section of the inserted support and valve part combination seen in FIG. 5, but rotated ninety degrees in a second orientation about a longitudinal axis.

FIG. 5B is a cross section of the valve part, seen in FIG. 9, at a plane of intersection between the inserted support and valve part.

FIG. 5C is a magnified cross section of the circled portion of FIG. 5A.

FIG. 6 is a cross section of a valve part which is similar to the valve part seen in FIG. 3, but comprising a modified distal front end.

FIG. 6A is a cross section of the valve part seen in FIG. 6, but rotated ninety degrees about a longitudinal axis.

FIG. 6b is an elevation of the distal end of the valve part seen in FIG. 6.

FIG. 7 is a cross section of an insertion support which is an integral part of a tapered fitting valve made with the valve part seen in FIG. 6.

FIG. 7A is a cross section of tha insertion support seen in FIG. 7, but rotated ninety degrees about a longitudinal axis.

FIG. 7B is an elation of the distal face of the insertion support seen in FIG. 7.

FIG. 8 is a cross section of the valve part seen in FIG. 6 with an insertion support as seen in FIG. 7 disposed therein.

FIG. 8A is a cross section of the inserted support and valve part seen in FIG. 8, rotated ninety degrees about a longitudinal axis.

FIG. 8B is a cross section at a plane of intersection between interfacing portions of the insertion support and an asymmetric portion of the assembled valve

FIG. 9 is a cross section of an assembled tapered fitting valve, as seen in FIG. 5, inserted into a female luer fitting.

FIG. 9A is a cross section of the assembled tapered fitting valve inserted into a female luer fitting as seen in FIG. 9, but rotated ninety degrees about a longitudinal axis.

FIG. 9B is a cross section of the valve part, seen in FIG. 9, at a plane of intersection between the insertion support and valve part.

FIG. 10 is a cross section of an assembled tapered fitting valve, as seen in FIG. 6, inserted into a female luer fitting.

FIG. 10A is a cross section of the assembled tapered fitting valve inserted into a female luer fitting as seen in FIG. 10, but rotated ninety degrees about a longitudinal axis.

FIG. 10B is a cross section of the valve part, seen in FIG. 10, at a plane of intersection between the insertion support and valve part.

FIG. 11 is a cross section of a male adapter which utilizes parts of the assembled valve seen in FIG. 5.

FIG. 11A is a cross section of the male adapter seen in FIG. 19, but rotated ninety degrees about a longitudinal axis.

FIG. 12 is an exploded view of parts (with portions in cross section) which when assembled combine to provide a medical syringe with an integrally affixed male adapter.

FIG. 13 is a magnified view of a circled portion of parts seen in FIG. 12.

FIG. 14 is an exploded view of the parts seen in FIG. 21 with a first valve part affixed to a medical syringe which has an insertion support integrally molded therewith.

FIG. 14A is a magnified view of a circled portion of the parts seen in FIG. 14.

FIG. 15 is a side elevation, with portions in cross section, of a completely assembled medical syringe and integrally affixed male adapter.

FIG. 15A is a magnified view of a circled portion of the parts seen in FIG. 15.

FIG. 16 is a side elevation, with a portion in cross section, of a male adapter with a cap disposed to cover and protect an otherwise exposed portion of a male adapter.

FIG. 17 is a cross section of a cap disposed as about an exterior of a male luer lock portion of a male adapter device comprising a preferred embodiment valve part.

FIG. 18 is a cross section of a cap and a male adapter before the cap is fully engaged about a luer lock portion of the adapter.

FIG. 18A is a cross section of parts which are similar to parts seen in FIG. 18 with a cap fully engaged and affixed to the male adapter.

FIG. 18B is a frontal elevation of a cap for a plurality of male luer lock fittings such as those, for example, seen in FIGS. 17, 18 and 18A, the interior surface of the male luer lock fitting contacting portion of the cap formed as an ellipse to match a similarly shaped luer lock fitting exterior and. thereby, provide instructing orientation for affixing the cap to the luer lock fitting.

FIG. 19 is a cross section at a plane of intersection between a valve part and an insertion support of a prior first valve part as disclosed in Thorne 343.

FIG. 19A is a schematic of the prior first valve part seen in FIG. 17 after being inserted into and compressed by a female luer fitting.

### DETAILED DESCRIPTION OF THE ILLUSTRATED EMBODIMENTS

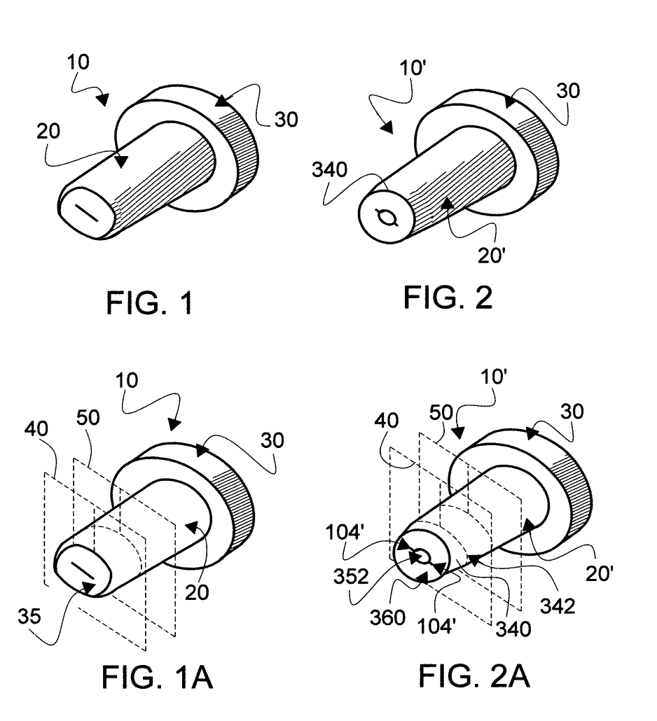

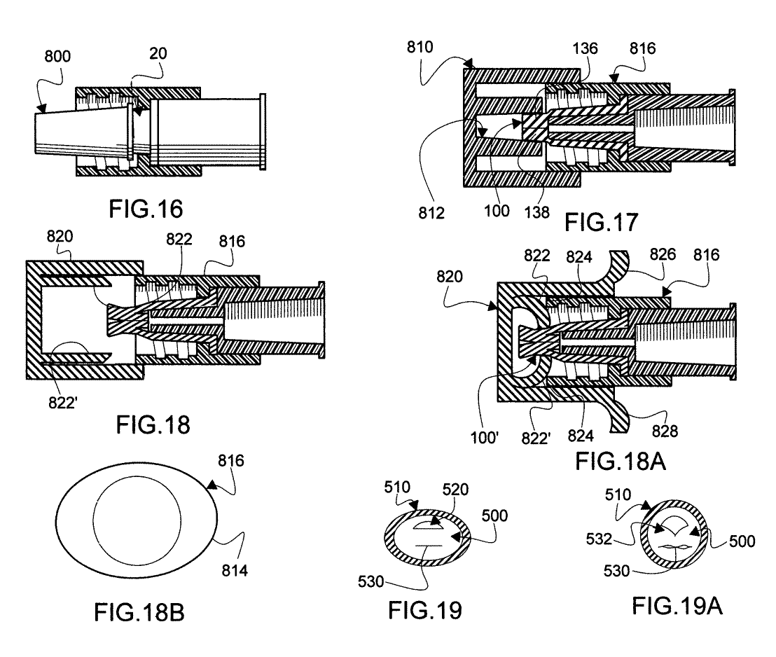

While the instant inventions disclosed herein are applicable to a wide variety of tapered male/female insertion type fluid connectors, the detailed description provided herein is focused upon examples of medical devices. Reference is now made to the embodiments illustrated in FIGS. 1-19B wherein like numerals are used to designate like parts throughout and primes of numbers generally indicate parts which are similar in shape and/or function of those numbers, but not exactly the same.

### Valve Part 10 (Preferred Embodiment)

Reference is now made to FIGS. 1 and 2 wherein an asymmetric valve part 10 is seen. As seen in FIG. 1, valve part 10 comprises two sections, an asymmetric, tapered body 20 and a proximally disposed asymmetric anchor ring 30.

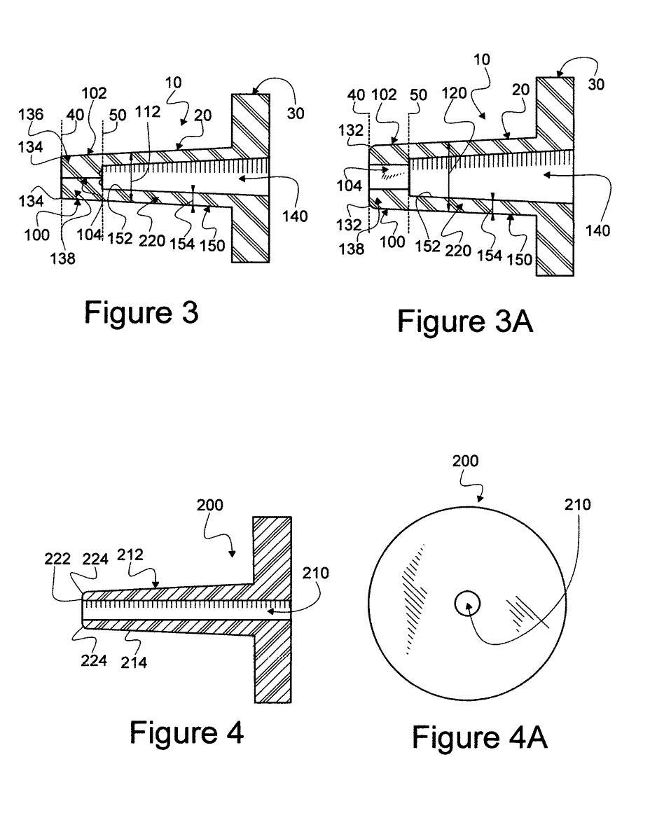

As seen in FIG. 2, at a distal end 35, a first reference orthogonal plane 40 is disposed to provide a cross reference definition. A second reference orthogonal dissecting plane 50 is disposed proximally from plane 50 to provide a second plane of definition. Plane 40 is disposed to define a distal end of a valve core 100 (details of which are not seen in FIG. 2, but seen in FIGS. 3 and 3A). Valve core 100 comprises a slit valve 102 formed by a planar slit 104 which is opened by radial compression of a planar slit 110.

Plane 50, as seen in FIGS. 3 and 3A, is disposed at the proximal end of valve core 100. Body 20, being elliptical in this example, is seen to be smaller in cross section in FIG. 3, which depicts a view about the minor axis 120 of the ellipse, than in FIG. 3A which depicts a view about the major axis 130 of the aforementioned ellipse. Each axis varies in length along a horizontal axis of part 10 as defined by taper of a fitting into which part 10 is displaced to open valve 102.

As seen in FIG. 3A, valve core 100 comprises a pair of beveled edges, commonly numbered 132, proximally disposed relative to plane 40. It should be noted, as seen in FIG. 3, that valve core 100 comprises a pair of sharp edges 134. The purposes for contour of edges 132 and 134 are explained in detail hereafter. As seen in FIG. 3, slit 110 forms a pair of lips 136 and 138.

Valve core 100 and body 20 combine to form a blind hole 140 which is also elliptically dimensioned as seen in FIGS. 3, 3A and 3B. Radial dimensions of hole 140 is defined by an exterior surface 150 of body 20, an interior surface 152 and at wall thickness 154. Exterior surface 150 is dimensioned to have a comparable circumference to the tapered fitting into which part 10 is inserted along the entire length of insertion. Wall thickness 154 is constant, the measurement of which is determined by dimensional limitations of the selected tapered fitting, as disclosed in detail hereafter.

### Valve Insert Support 200

A valve insert support 200 is seen in FIGS. 4 and 4A. Support 200 basically has three functions. First, support 200 provides a fluid communicating through flow pathway 210 for fluid communication to valve core 100 once support 200 is inserted into valve part 10. Second, support 200, once inserted, provides physical support for valve part 10 when both part 10 and support 200 are further inserted into the associated fitting. As valve part 10 is generally made from material which is subject to deformation such support is required. Third, support 200 comprises an insertion stem 212, which comprises an elongated circular structure 214 which is sized and shaped to reform that portion 220 of body 20 (see FIGS. 3 and 3A) into which stem 212 is inserted from elliptical to circular (i.e. to match structure of a female tapered fitting). Valve support 200 has yet one other very important function. At a distal end 222, where valve support 200 interfaces with valve core 100, valve stem 212 comprises a beveled end 224, which provides a reduction in stress about surface 152 where stem 212 and valve core 100 merge.

### Valve Assembly 300

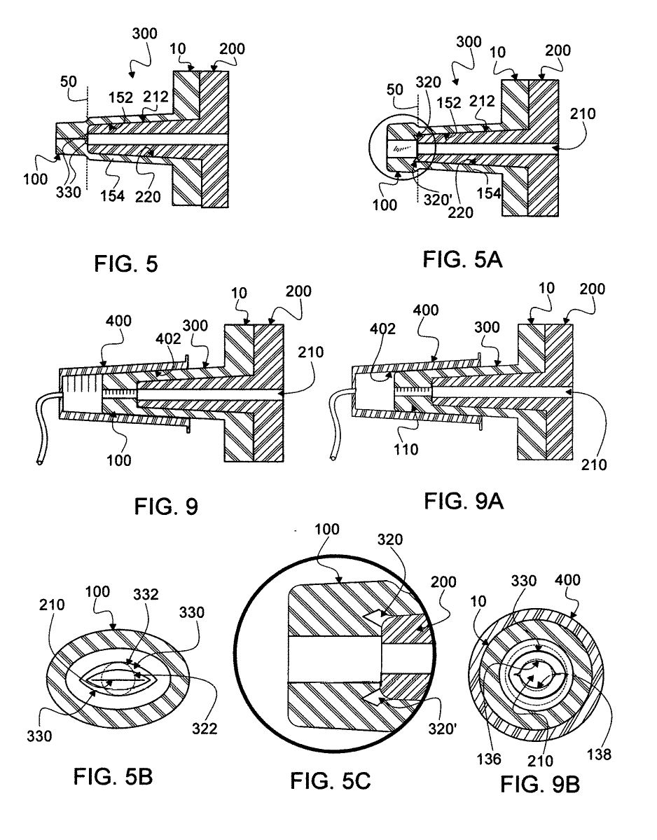

Reference is now made to FIGS. 5 and 5A wherein an assembled valve 300 is seen. Insertion of support 200 transforms a surrounding body portion 220 of valve part 10 from an elliptical to a circular cross section. Support stem 212 is sized to engage the inner surface 152 of body portion 220 in a fluid tight relationship, as seen in FIGS. 5 and 5A.

When so assembled and not inserted into a tapered fitting which opens valve 100 by radially directed ellipse deformation, valve part 10 must remain closed to fluid flow in both directions. When upstream pressure is less than ambient surrounding pressure, valve 100 performs as a conventional duckbill valve, remaining closed due to externally existing atmospheric pressure.

When upstream pressure is greater than ambient, it is well understood by those skilled in fluid dynamics that body portion 220 could expand and such expansion could part lips 136 and 138 with resultant valve opening. It should be noted that insertion of stem 212 into body 20 should result in a very tight fit about the minor elliptical axis 112 of body 20 as seen in FIG. 5.

Such is not the case about the major elliptical axis 120 near valve 100. As seen in FIG. 5A, insertion of stem 212 into body portion 220 also reforms wall 154 to be circular in cross section. However, the length of the major axis of the ellipse is greater than the diameter of the stem at the interface between valve 100 and stem 212. The result is a pair of open gaps 320 and 320′, seen in FIG. 5A and better seen by magnification in FIG. 5C. By providing a fluid communicating pathway into gaps 320 and 320′, body 20 tends to expand about major elliptical axis 120. Such expansion tightens the fit about minor axis 112 (see FIG. 5) while such expansion lengthens body 20 along major axis 120 (see FIG. 3A) and thereby tightens contact between lips 136 and 138 acting to keep valve 100 closed.

To provide a fluid pathway which communicates fluid and associated pressure via hole 124 to gaps 320 and 320′, a pattern of grooves, generally numbered 330, are disposed in the proximal surface portion 322 of valve 100, as seen in FIG. 5B. Note that groove pattern 330 (seen in FIGS. 3, 5 and 5B) is disposed to communicate with pathway 210 (denoted by dashed lines in FIG. 5B). the location of which is defined by a dashed line circle 332.

### Valve Part 10′

Reference is now made to FIGS. 1A and 2A wherein an asymmetric valve part 10′ is seen. Valve part 10′ comprises three general sections, a distal insertion end and transition zone 340, an elliptically shaped body 20′ and an anchor ring 30.

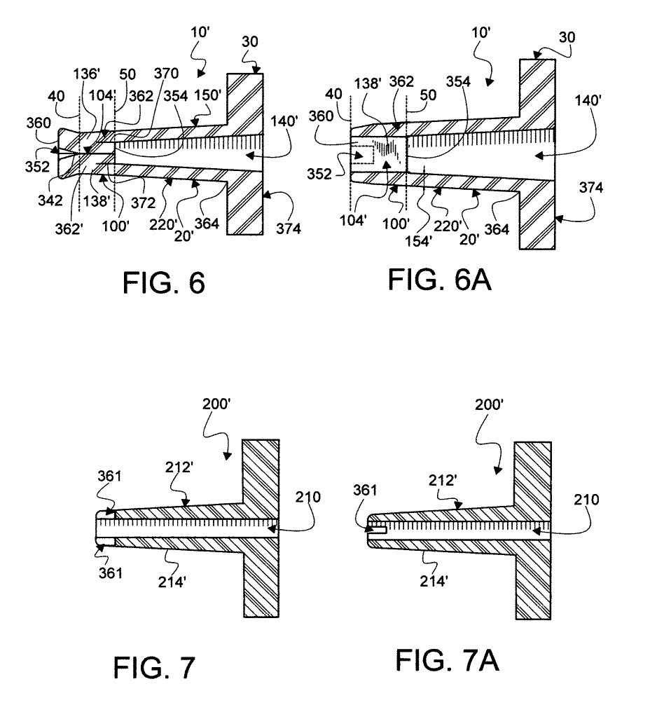

As seen in FIG. 2A, a first reference orthogonally dissecting plane 40 is disposed proximally from zone 340. A second reference dissecting plane 50 is disposed proximally from plane 40. Plane 40 is proximally disposed relative to section 340 at a site which defines a proximal end 342 of a transition section 350 between end 342 which is the distal end′ of a valve 100′ (not seen in FIG. 2A, but seen in FIGS. 6 and 6A).

Distal portions of a slit 104′ which cleaves through end 342 and valve core 100′ (to form a pair of lips 136′ and 138′ as seen in FIG. 6) is seen to extend on opposite sides of a blind hole 352 which is circular at end 360, see FIG. 2A, and diminishes linearly to closure along slit 104′ at plane 40 between lips 136′ and 138′, as seen in FIG. 6. Plane 50 is disposed to reference the proximal end 122 of valve 100′, which is also the proximal end of two back-to-back slit (duckbill valves), numbered 362 and 362′ as seen in FIGS. 6 and 6A.

Generally, the proximal exterior surface 150′ of valve core body 20′ is shaped to form an ellipse which is tapered proximally to conform with the 3° taper of a luer fitting. Because end 360 is circular, section 340 (see FIG. 2A) provides a linear transition from a circle to the shape of the ellipse of the rest of body 20′ of part 10′, while keeping cross sectional surface circumference of section 340 equal to an inner surface circumference of a corresponding surface of a female luer fitting into which part 10′ is fully inserted. From plane 40, to the proximal end 364 of part 10′, the exterior surface 150′ of body 20′, as disclosed supra, is elliptical and conforms to a 3° taper. Such a smooth contour is uniquely different than embodiments of similar valves disclosed in U.S. patent applications from which this application continues. Other marked differences are two blind slits, numbered 370 and 372, also disposed in valve core 100′.

A blind, tapered hole 140′ which opens through proximal end 374 ends at valve core 100′, as seen in FIGS. 6 and 6A. Blind hole 140′ is sized to be proportionally smaller than surface 150′ to provide a constant wall thickness 154′ along the length of blind hole 140′ through body 20′. The elliptical shape of hole 140′ is maintained in proximal end anchor ring 30, as seen in FIGS. 6 and 6A.

Distal portions of slit 104′ which cleaves through valve core 100 (to form lips 136′ and 138′) is seen to extend on opposite sides of a blind hole 352 which is circular at end 360, see FIGS. 2A and 6B, and diminishes linearly to closure at slit 104′ at plane 40 between lips 136′ and 138′, as seen in FIGS. 6 and 6A. Form and structure at end 360 is seen in FIG. 6B. Blind hole 352 is terminated at Plane 40 along slit 104′. Plane 50 is disposed to reference the proximal end 354 of valve core 100′.

### Valve Insert Support 200′

A valve insert support 200′ is seen in FIGS. 7. 7A and 7B. Support 200′ basically has the same three functions as support 200, but relative to part 10′, disclosed supra. Repeating, first, support 200′ provides a fluid communicating through pathway 210 for fluid communication to valve core 100′ once support 200′ is inserted into valve part 10′. Second, support 200′, once inserted, provides physical support for valve part 10′ when both part 10′ and support 200′ are further inserted into an associated tapered fitting. As valve part 10′ is generally made from material which is subject to deformation such support is required. Third, support 200′ comprises an insertion stem 212′, which comprises an elongated circular structure 214′ which is sized and shaped to reform that portion 220′ of body 20 (see FIGS. 6 and 6A) into which stem 212′ is inserted from elliptical to circular (i.e. to match structure of a female tapered fitting). Valve support 200′ has yet one other very important function. At a distal end 222 (see FIG. 4), where valve support 200 interfaces with valve core 100, valve stem 212′ comprises a beveled end 224, which provides a reduction in stress about surface 152 where stem 212 and valve core 100 merge and an open notch 361 thereat, which is also seen in FIG. 7B.

### Valve Assembly 300′

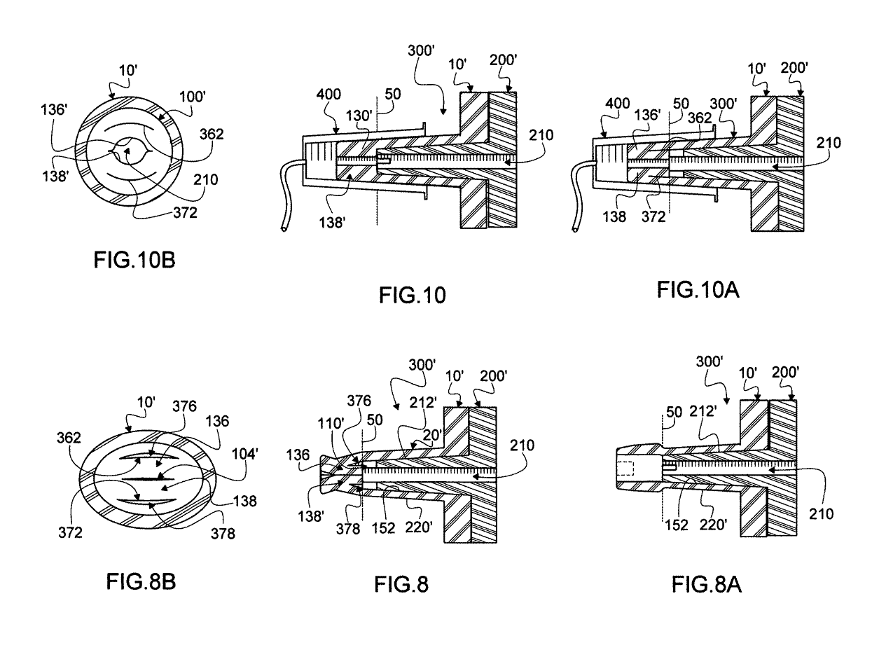

Reference is now made to FIGS. 8 and 8A wherein an assembled valve 300′ is seen. Insertion of support 200′ transforms a surrounding body portion 220′ of valve part 10′ from an elliptical to a circular cross section. Support stem 212′ is sized to engage the inner surface 152 in a fluid tight relationship, as seen in FIGS. 8 and 8A.

When so assembled and not inserted into a tapered fitting which opens valve 100′ by radially outward directed deformation, valve part 10′ must remain closed to fluid flow in both directions. When upstream pressure is less than ambient surrounding pressure, valve 100′ performs as a conventional duckbill valve, remaining closed due to externally existing atmospheric pressure.

When upstream pressure is greater than ambient, it is well understood by those skilled in fluid dynamics that body portion 220′ could expand and such expansion could part lips 136′ and 138′ with resultant valve opening. It should be noted that insertion of stem 212 into body 20′ should result in a very tight fit about the minor elliptical axis 112′ of body 20′ as seen in FIGS. 8 and 8A. In the case of assembled valve 300′, the tight fit operates to displace blind slits 362 and 372 (see FIG. 8B) to an open state creating blind cavities 376 and 378 as seen in FIG. 8.

As seen in FIGS. 7 and 7A, stem 212′ comprises open end notch 361 which permits fluid and fluid pressure to communicate with cavities 376 and 378. Such communication of fluid pressure places the same pressure about lips 136′ and 138′ resulting in no additional valve opening forces.

Inserting Valves 300 and 300′ into a Female Luer Fitting

Reference is now made to FIGS. 9, 9A and 9B wherein a valve 300, as an example, is disposed within a female luer fitting 400. It should be noted that support 200 should be constrained to remain within valve part 10. However, in FIGS. 9 and 9A constraining members are not shown to reduce complicating structures and permit a clearer presentation of valve 300 performance within a female luer fitting. Examples of devices, each employing valve 300 and a constrained support 200, are provided hereafter.

Fitting 400 is a conventional tapered luer fitting having a circular cross section. As shown in FIGS. 9 and 9A, fitting 400 compresses valve core 100 to open a through hole (which is then a continuation of pathway 210 and, therefore, given the same number. A fluid tight fit is assured by constructing each linear circumferential segment of valve 300 to have the same circumference as the corresponding inner surface 402 of fitting 400. Exemplary geometry of pathway 210 which is opened between lips 136 and 138 is seen in FIG. 9B. Note, that associated parting of lips 136 and 138 also displaces groove pattern 330 away from pathway 210. Assurance of opening of pathway 210 thereat is provided by a medially disposed slit 110.

Evidence of lack of enablement of fittings disclosed in prior U.S. patent applications from which this U.S. patent application continues-in-part is provided in FIGS. 19 and 19A. FIG. 19 discloses a proximal end 500 of a valve core 510, similar in desired operation to valve core 100 of the present invention. However, the prior applications taught a molded cavity 520 disposed to provide fluid pressure upon a slit, numbered 530 in this example. It was anticipated that cavity 520 would close upon radially directed compression occurring when disposed in a female luer fitting. But is was discovered that, rather than being compressively closed, structure 532 about cavity 520 was distorted as seen, by example, in FIG. 19A. Such distortion effectively kept slit 530 from opening.

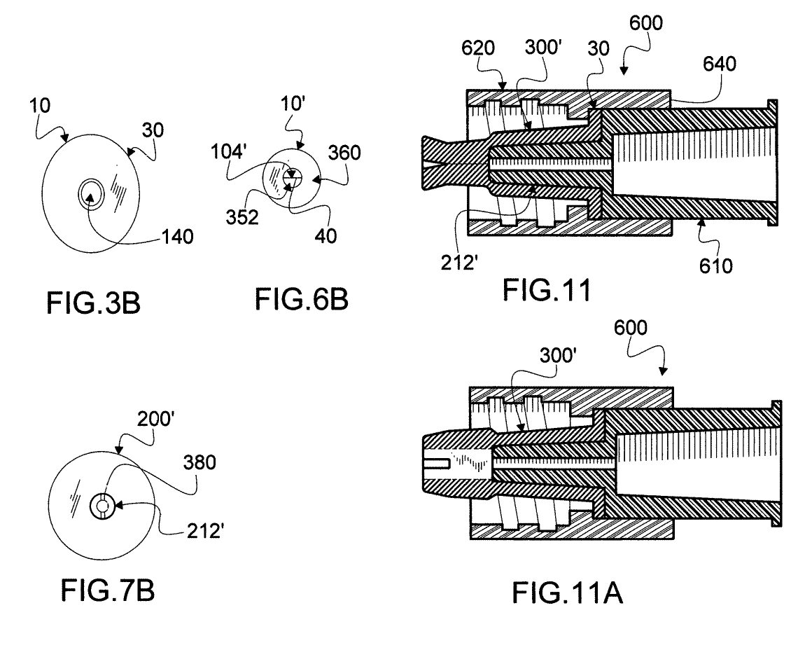

As seen in FIGS. 10, 10A and 10B, a valve 300′ is inserted into female luer fitting 400. Note, in FIGS. 10A and 10B, that slits 362 and 372 are closed. Flow path 210 is extended by parting lips 136′ and 138′.

### Critical Dimensions of Valve Part 10 (and 10′)

Dimensions of major and minor axes of part 10 ellipse are dependent upon the diameter of a fluid pathway 210 formed by radial compression when part 10 is inserted into a conventional luer fitting 400 (see FIGS. 9 and 9A). As an example, if a fluid pathway 210 has a predetermined diameter and the distance along lips 136 and 138 (valve length) is of the order of 0.110 inches. In the case of part 10′, transition section 340 (see FIG. 2A) transition length may be 0.050 inches, although not of consideration in this example. With the aforementioned dimensions, the following calculated parameters (in inches) of part 10 apply:

| | For a.050 | For a 0.60 |
|---|---|---|
| Item | dia. hole | dia. hole |
| Valve length | 0.110 | 0.110 |
| Total body 20 length | 0.400 | 0.400 |
| Calculated slit 110 width | 0.079 | 0.094 |
| Slit width extended for open orifice | 0.083 | 0.099 |
| anomalies | | |
| Stem pathway 210 hole diameter | 0.050 | 0.060 |
| Support stem 212 dist end diameter | 0.109 | 0.109 |
| Support stem 212 distal end wall thickness | 0.030 | 0.025 |
| Support stem 212 bevel radius | 0.025 | 0.025 |
| Major (A) axis (at plane 40-with extended | 0.196 | 0.201 |
| slit) | | |
| Minor (B) axis (at plane 40-with extended | 0.109 | 0.099 |
| slit) | | |
| Ellipse A axis (at plane 50-with extended | 0.207 | 0.213 |
| slit) | | |
| Ellipse B axis (at plane 50-with extended | 0.119 | 0.109 |
| slit) | | |
| Ellipse A axis (at proximal end of body 20) | 0.251 | 0.257 |
| Ellipse B axis (at proximal end of body 20) | 0.161 | 0.151 |

Calculations of A and B axes are made at reference plane 50 as follows:

half axis b=ILFR−HR

B axis−2*b

half axis a=Sqrt(2*HR²−b²)

A axis=2*a

### Where:

A is major ellipse axis and “a” is major half axis

B is minor ellipse axis and “b” is minor half axis
Note: A and B axes are thus calculated to provide a circumference equal to the internal circumference of a female luer fitting at a site at which part 10 is fully inserted. In other words, the female luer diameter which correlates to plane 50 is 0.176 inches with a circumference of 0.552 inches; the female luer diameter which correlates to plane 60 is 0.193 inches with a circumference of 0.605. inches.

ILFR is internal luer fitting at reference plane radius

SL is actual slit length

HR is desired pathway 210 hole radius

Sqrt is square root

As mentioned supra, slit 104 width can be calculated as one-half pi times desired hole diameter, but a differential circumference from that calculated for a circular hole resulting from shape variations at slit 110 ends suggests an small increase to slit length be added. In the calculations above, a five percent increase to calculated slit length has been added.

An additional calculation to assure meeting pathway 210 desired orifice size of the above listed parameters (i.e. ellipse area against area of fitting at a common plane shows the following:

| | | |
|---|---|---|
| Area (at plane 40) | 0.0187 | 0.0184 |
| Percent less than area of fitting at plane | 5 | 7 |
| Area (at plane 50) | 0.0213 | 0.210 |
| Percent less than area of fitting at plane | 5 | 6 |
| Area (at proximal end of body 20) | 0.0337 | 0.333 |
| Percent less than area of fitting at plane | 4 | 5 |

Thus, with the parameters provided supra, a larger pathway 210 cross section can be generated than hole size specified.

As mentioned supra, one of the critical issues associated with luer fitting design according to the present invention is meeting ISO standards. For this reason, distal end 35 must be consistent with a limited circular orifice and, therefore, limited to a circular insertion diameter of 0.158 inches. Therefore edges 132 and 134 see FIG. 3A) to meet ISO standards.

Male Adapter 600 Utilizing Elements of Valve Assembly 300 (i.e. Valve Part 10 and a Stem 212)

An exemplary male adapter 600 which employs inventive elements of valve assembly 300′ (see FIGS. 8 and 8A) is seen two rotational modes in FIGS. 11 and 11A. Adapter 600 comprises a valve part 10′, a female luer fitting 610 which comprises an integrally molded stem 212′ and a male luer lock fitting 620. Fittings 610 and 620 are joined along a common interface 640 by compression, adhesion, welding or threading (all commonly used in medical device construction) to capture anchor ring 30 (see FIG. 2). As such adapter 600 meets or exceeds all requirements for a self-closing fitting for medical applications.

### Syringe Application for Valve Part 10

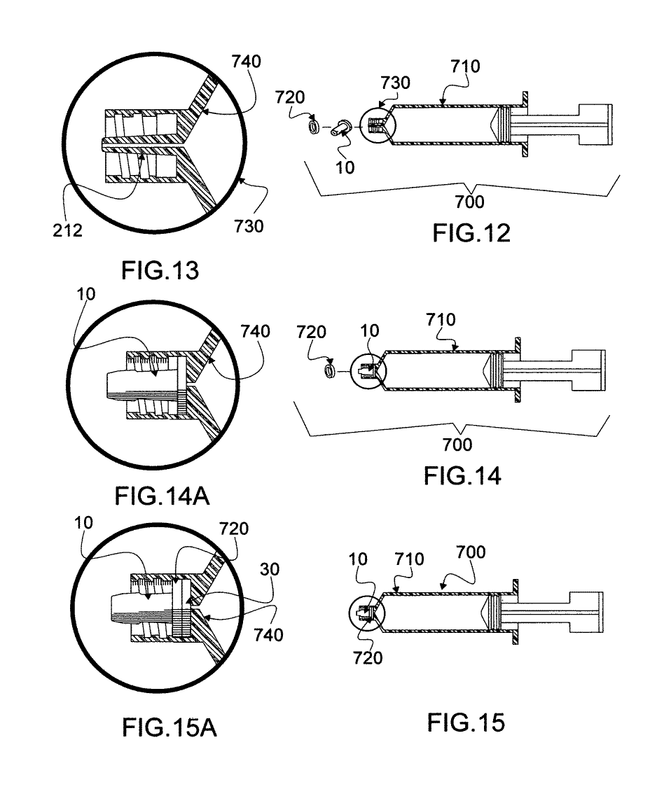

A syringe system 700 which employs a valve part 10 (and assembly 300) in place of a conventional male luer fitting is seen in various stages of assembly in FIGS. 12-15A. As seen in exploded format in FIG. 12, syringe system 700 comprises a conventional medical syringe 710 which is modified for interface with a valve part 10 and a retaining ring 720.

As seen in magnified circled reproduction 730 of a portion 740 of syringe 710, syringe 710 comprises an integrally molded stem 212 in place of a conventional male luer. Valve part 10 is affixed about stem 212 as seen in FIGS. 14 and 14A. As a final assembly step, retaining ring 720 is affixed to provide compressive, secure engagement as seen in FIGS. 15 and 15A.

### Caps

A variety of caps which can be used to protect a fitting made according to the present invention are seen in FIGS. 15-18B. As seen in FIG. 26, a simple conventional female luer cap 800 can also be used. However, such a cap must grip body 20 (or 20′) at a site which maintains valve part 10 in an open state.

Cap 810, seen in FIG. 17) comprises internal structure 812 which is sized and shaped to compress valve lips 136 and 138 closed. For cap 810 and another cap 820 (disclosed hereafter) to be safely and efficaciously used, exterior wall 814 of associated luer fitting 816 must be formed to radially orient caps 810 and 820. An example, of such is seen in FIG. 18B where wall 814 is seen to be asymmetric (e.g. elliptical).

The cap 820 which is designed to maintain closure pressure upon a thinned or minor axis portion of valve core 100 is seen in FIGS. 18 and 18A. Cap 820 is made from a substantially incompressible yet flexible material. As seen in FIG. 18, cap 820 comprises a pair of internally disposed leaflets 822 and 822′. As cap 820 is disposed about a male luer lock fitting 816, leaflets 822 and 822′ are forced inward by collision with distal structure 824 of the male luer fitting 816 to engage and apply pressure upon valve core 100′ as seen in FIG. 18A. Note in FIG. 18A, a pair of flanges 826 and 828 are added to cap 820 to facilitate engagement with and removal from fitting 816. As mentioned supra, for cap 820 to be used, proper orientation is necessary.

Inventions disclosed herein may be embodied in other specific forms without departing from the spirit or essential characteristics thereof. The present embodiments are therefore to be considered in all respects as illustrative and not restrictive, the scope of the inventions being indicated by the appended claims rather than by the foregoing description, and all changes which come within the meaning and range of equivalency of the claims are therefore intended to be embraced therein.

What is claimed and desired to be secured by Letters Patent is:

1. A normally-closed male valve which is opened upon insertion into a tapered female fitting of circular cross section, said valve comprising:
(i) a valve part comprising
a) a distally disposed planar surface;
b) a tapered asymmetric valve core section comprising a distal end, a proximal end and a planar slit there between;
c) an extended, hollow body, comprising a comparable tapered asymmetric shape as said valve core section, proximally affixed to said proximal end of said valve core section, and
d) an anchor ring integrally affixed at the proximal end of the extended body;
said valve core section comprising an exterior surface which comprises the same relative circumferential dimensions as an internal surface of the tapered female fitting, when fully disposed therein, said slit having a width dimension which, when opened by radial compression of said valve core section, defines a fluid pathway of predetermined flow capacity but when closed by relaxation of uncompressed material maintains closure of said valve; and
said extended body comprises an exterior surface which is increased in size to match the surface taper of the fitting cross section, when fully inserted therein; and
(ii) a tapered internal support which is displaced into said hollow body to provide structural support, said support further comprising:
a) an elongated stem comprising a tapered circular cross section which is displaced into said hollow body to reform the asymmetric shape of said hollow body to a conforming, close fitting, circular cross section comparable to that of the tapered fitting and further comprising a distal end structure by which fluid and fluid pressure is communicated to deter valve opening under force of upstream pressure.

2. A male valve according to claim 1 wherein said distally disposed planar surfaces fits within a circle of 0.158 inches.

3. A male valve according to claim 1 wherein said asymmetric valve core section comprises an elliptically shaped exterior surface.

4. A male valve according to claim 1 wherein said extended hollow body comprises an elliptical shape and a body wall of constant thickness.

5. A male valve according to claim 3 wherein said anchor ring is elliptically shaped with exterior dimensions which are proportional to those of said valve core section.

6. A male valve according to claim 3 wherein said elongated stem comprises distal end structure which is beveled to thereby provide a fluid pathway to gaps associated with a major axis of said elliptical shape.
